(19) United States
(12) Patent Application Publication
(10) Pub. No.: US 2012/0090613 A1
(43) Pub. Date: Apr. 19, 2012

(54) Breathing Apparatus

(75) Inventors: Kok Meng Woon, Singapore (SG); Ah Kat Tan, Singapore (SG); Kaya Totong, Singapore (SG); Teck Soon Ong, Singapore (SG); Geoffrey Chor Yong Tan, Singapore (SG)

(73) Assignee: Ngee Ann Polytechnic, Singapore (SG)

(21) Appl. No.: 13/260,137

(22) PCT Filed: Oct. 14, 2010
(86) PCT No.: PCT/SG2010/000391

§ 371 Date: Sep. 23, 2011

(51) Int. Cl. A61M 16/06

(52) U.S. Cl. 128/205.25

(57) ABSTRACT

A breathing apparatus comprising: an inlet configured to receive pressurised gases, an outlet configured to diffuse the pressurised gases within the dead space of a face mask, a detachable nozzle manifold configured to pass the pressurised gases from the inlet to the outlet, and a seal configured to substantially air tight seal the nozzle manifold between a user's face and a bottom inner surface of the mask.

### FIELD OF THE INVENTION

The present invention relates to a breathing apparatus.

### BACKGROUND OF THE INVENTION

Disposable face masks are used in a number of applications to filter the air that people breathe. This may be used to prevent the passage of air borne pathogens either to or from the lungs, or may filter larger particles such as pollutants.

Such masks may be worn by elderly people or those suffering from respiratory problems to protect the weakened immune system from air borne infections. Healthcare professionals use such masks to prevent the spread of infections in healthcare facilities. Frequent travellers such as business people may wear such masks in high infection risk locations such as pressurised jet aircraft.

Typically such masks may be worn for extended periods. As such a build up of moisture, carbon dioxide and/or filtered particles can accumulate. Because the pores of the filter material may become blocked over time, breathing may become more uncomfortable the longer the mask is worn. Such additional impediment and/or discomfort may not be desirable, especially for patients already experiencing breathing difficultly.

An example of such a disposable mask is an N95 certified mask. N95 is a certification by the National Institute for Occupational Safety and Health (NIOSH), for “occupation respirators” or “surgical respirators” where “N” means Not resistant to oil', and “95” refers to a 95% filter efficiency. They reduce the risk of the wearer from infecting others and to some extent filtering the air before it enters the respiratory tract. N95 masks are designed to filter 95% of particles (particulate aerosols free of oil) that are 0.3 microns in size or larger.

### SUMMARY OF THE INVENTION

In general terms the invention proposes a removable nozzle that seals within a mask to deliver pressurised gases. This may have the advantage that a standard disposable mask may not need to be modified, the nozzle may be disposable, the ease of breathing may be improved, moisture build-up within the dead space inside the mask may be reduced, the apparatus may be convenient and ergonomic and/or the apparatus may be low cost.

In a first specific expression of the invention there is provided a breathing apparatus according to claim 1. Embodiments may be implemented according to any of claims 2 to 9.

### BRIEF DESCRIPTION OF THE DRAWINGS

In order that the invention may be fully understood and readily put into practical effect there shall now be described by way of non-limitative example only, example embodiments described below with reference to the accompanying illustrative drawings in which:

FIG. 1(a) is front view of a disposable mask with a nozzle delivering filtered air according to a first example embodiment;

FIG. 1(b) is back view of the mask in FIG. 1(a);

FIG. 2 is front interior view of the air filter unit in FIG. 1(a);

FIG. 3(a) is front perspective view of the mask and nozzle in FIG. 1(a);

FIG. 3(b) is back perspective view of the mask and nozzle in FIG. 1(a);

FIG. 4(a) is a perspective view from the top of the nozzle in FIG. 1(a);

FIG. 4(b) is a perspective view from the bottom of the nozzle in FIG. 1(a);

FIG. 4(c) is top view of the nozzle in FIG. 1(a);

FIG. 4(d) is side view of the nozzle in FIG. 1(a);

FIG. 4(e) is front view of the nozzle in FIG. 1(a);

FIG. 5(a) is front perspective view of the mask and nozzle according to a second example embodiment;

FIG. 5(b) is back perspective view of the mask and nozzle in FIG. 5(a);

FIG. 6(a) is a perspective view from the top of the nozzle in FIG. 5(a);

FIG. 6(b) is a perspective view from the bottom of the nozzle in FIG. 5(a);

FIG. 6(c) is top view of the nozzle in FIG. 5(a);

FIG. 6(d) is side view of the nozzle in FIG. 5(a);

FIG. 6(e) is front view of the nozzle in FIG. 5(a);

FIG. 7 is a photo of the mask in use on a person;

FIG. 8 is a photo of a test apparatus for leaks; and

FIG. 9 is a photo of fit testing of the mask.

### DESCRIPTION OF EMBODIMENTS

A breathing apparatus 100 according to the first example embodiment is shown in FIGS. 1 to 4. An air filter unit 102 draws in ambient air, provides filtering and raises the pressure. A flexible conduit 104 carries the pressurised filtered air to the mask 106 fitted about the user's 108 nose and mouth. A nozzle 110 is connected at the end of the conduit 104 and provides a sealed interface for the pressurised filtered air into the dead space 112 within the mask 106.

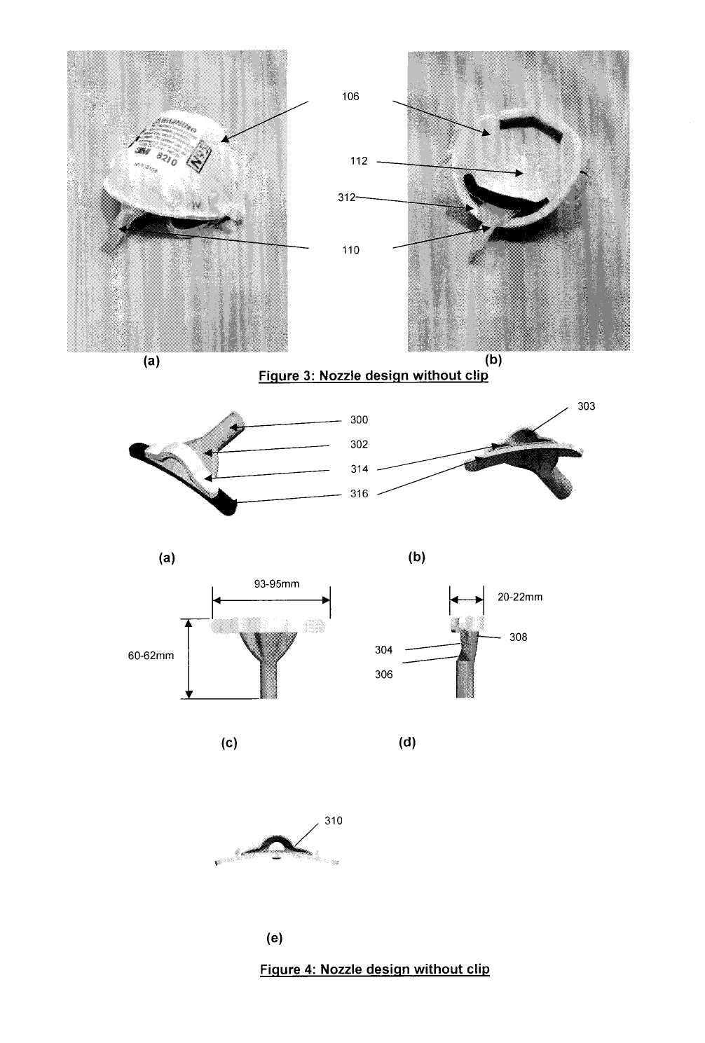

The nozzle 110 is shown in more detail in FIGS. 3 to 4. The nozzle 110 includes an inlet 300 of a suitable external diameter for a friction fit seal against the outlet of the conduit 104. A nozzle manifold or nozzle body 302 diffuses the air from the inlet 300 to a much wider outlet 303. It is desirable to diffuse the air to avoid it being blown directly up the user's nose or causing irritation or discomfort. The inner side 304 of the manifold 302 includes a scallop 306 designed to follow the contour of an average user's chin. The outer side 308 includes a curved profile 310 designed to follow the bottom inner surface 312 of the mask 106. An inner seal 314 seals between the scallop 306 and the user's chin. An outer seal 316 seals between curved profile 310 and the bottom inner surface 312. The nozzle is made from medical grade polymer.

The shape of the outlet 303 is designed with the necessary minimum cross-sectional area of 80 square millimetres, i.e. the cross-sectional area of the conduit 104, to allow 20-30 L/min of filtered air to pass through; and at the same time ensuring the effectiveness of the inner seal 314 and the outer seal 316. Thus, the maximum height of the opening of the outlet 303 is in the middle portion, the shape tapers gradually to the two ends and the two ends are sharp to provide effective sealing when the inner seal 314 and then outer seal 316 are put on.

The scallop 306 is designed with a curvature that follows the contour of an average adult user's chin. The 5-8 mm thickness of the outer seal 316 provides the flexibility and effectiveness of sealing for users with variations in chin contour. Initially, different rectangular and thicker sizes and shapes were designed and tested, but they failed the fit test (i.e. leakage test), until the scallop 306 design was arrived. For users with smaller chin size, e.g. children and teenagers, the radius of the scallop 306 is reduced accordingly to provide effective sealing.

The inner seal 314 and the outer seal 316 are foam rubber or sponge rubber attached with adhesive or glue to the manifold 302. The inner seal 314 is rectangular approximately 85-95 mm long, 10-15 mm wide and 5-8 mm thick. The outer seal 316 is rectangular approximately 60-70 mm long, 10-15 mm wide and 5-8 mm thick. The seals are rectangular in cross-section so that they can be easily manufactured by cutting out from a large piece of standard foam rubber or sponge rubber material.

The bottom inner surface 312 may be used for the nozzle 110 to attach to the mask 106. This may be advantageous because it may avoid any torsion or twisting on the nozzle that might occur if it was located on the side of the mask. This may avoid any leaks caused by the mask edge being lifted and any kinks in the conduit.

The conduit 104 runs from the air filter unit 102 to the nozzle inlet 300. It is constructed on medical grade silicone with an inner diameter of 8 mm and a wall thickness of 1 mm. The length depends on the size of the user and is typically 300-500 mm long. The conduit 104 may be clipped to or worn underneath a user's clothing.

A quick release connector 212 is provided between the outlet of the air filter unit 102 and the inlet of the conduit 104 for easy connection and disconnection of the conduit 104.

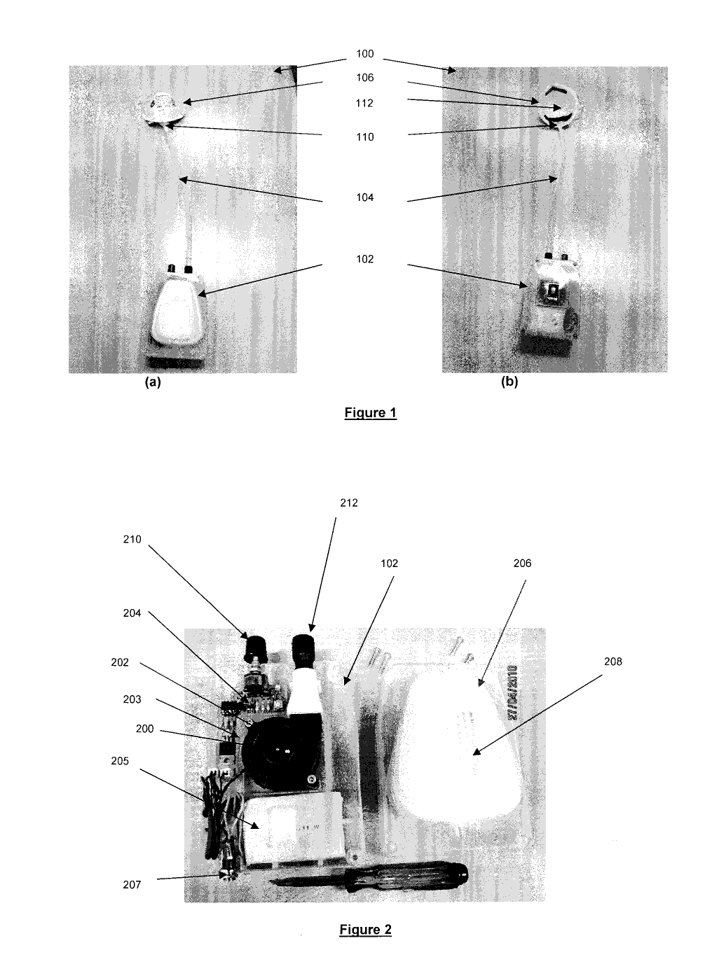

The air filter unit 102 is shown in more detail in FIG. 2. It includes a brushless DC motor 200 driving a centrifugal fan 202. The motor 200 is energised by an electronic controller 204 which is powered by an 11.1V 1600 mAh Lithium Polymer rechargeable battery 205. For normal use the air filter unit 102 delivers approximately 20-30 L/min of filtered air for up to 4 hours use. The air filter unit 102 is made of medical grade polymer and includes a belt clip for attachment.

The inlet to the fan 202 is sealed and covered with a snap on cover 206 for a flat filter 208. The filter may be compliant with the same standard as the mask 106, such as N95. The snap on cover 206 allows easy changing of the flat filter 208 when required.

The electronic controller 204 includes a PCB, and a control knob 210. The user can rotate the control knob to select what speed the fan runs at. The electronic controller 204 is in a separate compartment 203 from the motor 200 and fan 202 which are sealed to ensure no contamination. A plug 207 is provided for an external battery charger to charge the battery 205. The motor is energised using pulse width modulation (PWM) for speed control. The combination of a brushless DC motor and PWM may provide high energy efficiency and/or longer battery life.

The majority of air within the mask 106 has been drawn in through filter 208, and not through the mask 106. This means the mask 106 gets choked less easily and thus lasts longer. In addition, the positive pressure within the mask provided by the invention increases the efficiency of breathing by: (1) increasing the speed of the exhaled air from the user to pass through the mask 106 to the surroundings, and (2) reducing the amount of pathogens and dirt sticking onto the outside of the mask 106. The invention has been tested by a few users and each of them found that the flow of filtered air from the apparatus in the mask 106 has significantly improved the ease and comfort of breathing.

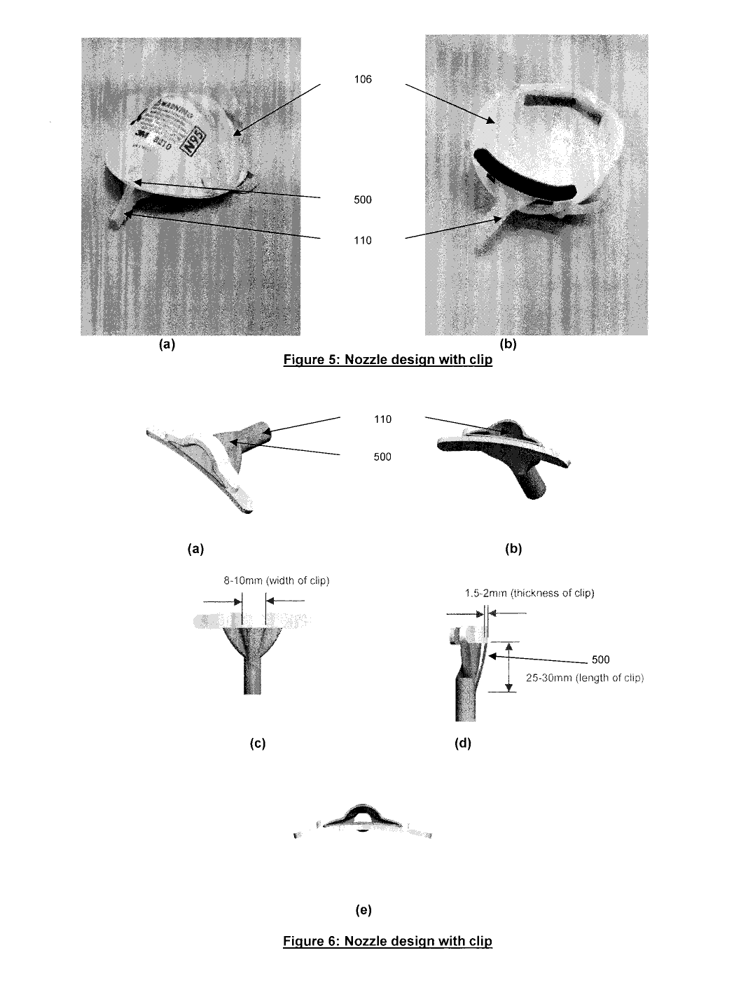

In FIGS. 1 to 4 the nozzle 110 is affixed via the outer seal 316 to the bottom inner surface 312 by adhesive, such as an adhesive tape or glue. Alternatively as shown in FIGS. 5 to 6, the nozzle 110 is affixed to the bottom inner surface 312 via a clip 500 according to a second example embodiment. In this case the clip 500 is 25-30 mm long, 8-10 mm wide and 1.5-2 mm thick, and is thus resilient such that it can be affixed to and removed from the mask 106 conveniently. Polymer materials based on polystyrene or polypropylene may provide the resilience needed.

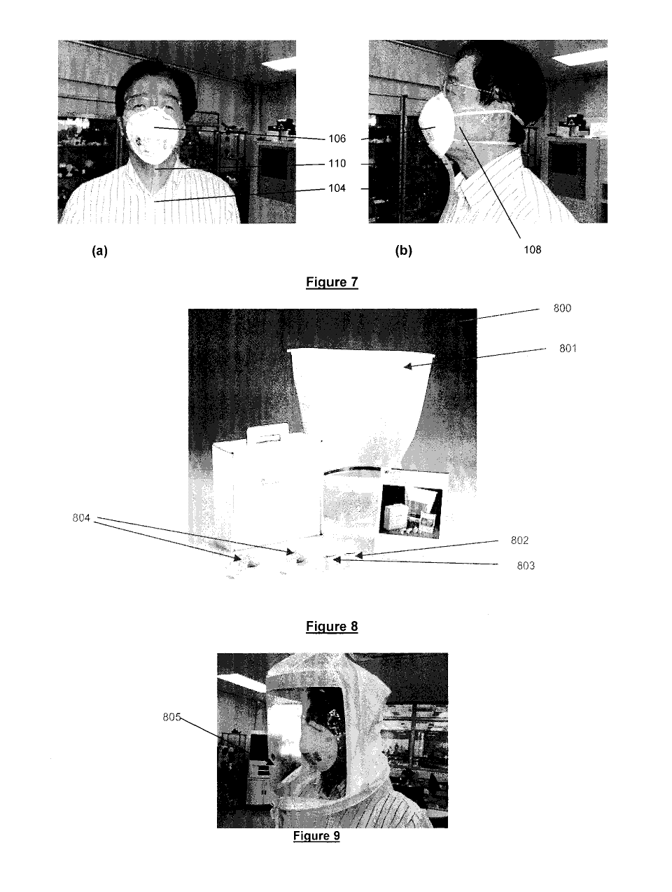

In FIG. 7 the mask 106 is shown in use attached to the face 108 of a user. As can be seen the nozzle 110 is at the bottom of the mask, and the conduit 104 follows comfortably under the chin and down the user's torso.

To check the sealing effectiveness of the nozzle 110, a 3M Qualitative FT-30 Fit Test Kit 800 was used, as shown In FIG. 8. The Test Kit comprises the Hood 801, a 55 ml bottle of the bitter Sensitivity Solution 802, a 55 ml bottle of the bitter Fit Test Solution 803, and 2 Nebulisers 804. The Sensitivity Solution is a very dilute version of the Fit Test Solution. During the fit test, as shown in FIG. 9, the Hood 801 is placed onto the user. According to the required procedure, the sensitivity test is first carried out to check whether the user is able to detect the bitter solution used. In this sensitivity test, the user does not wear the mask with nozzle. Two or three drops of the Sensitivity Solution 802 are dropped into one of the Nebulisers 804. An aerosol of the Sensitivity Solution 802 is then introduced into the Hood 801 from the Nebuliser 804 via a hole 805 at the front of the Hood 801. If the user is not able to detect the bitter taste, then another user will be needed for the sensitivity test. Once a suitable user is available, the subsequent actual Fit Test follows, in which the user puts on the mask 100 with the nozzle 110, and introduces an aerosol of the Fit Test Solution 803 using the other Nebuliser 804.

The sealing effectiveness using a 3M Qualitative FT-30 Fit Test Kit 800 has been tested for both nozzle designs, i.e. nozzle without clip (FIGS. 3 and 4) and with clip (FIGS. 5 and 6), and both designs passed the fit tests.

While various example embodiments have been described in the detailed description, it will be understood by those skilled in the technology concerned that many variations in details of design, construction and/or operation may be made without departing from the scope as claimed. For example the air filter unit may be integrated into the nozzle and the conduit dispensed with. The power source may be separated from the air filter unit and may provide power generation such as from the movement of the user or from solar panels. The nozzle may be used for delivering other kinds of gases such as higher oxygen mixes and/or can be used with non disposable masks and breathing equipment.

1. A breathing apparatus comprising:
an inlet configured to receive pressurised gases,
an outlet configured to diffuse the pressurised gases within the dead space of a face mask,
a detachable nozzle manifold configured to pass the pressurised gases from the inlet to the outlet, and
a seal configured to substantially air tight seal the nozzle manifold between a user's face and a bottom inner surface of the mask.

2. The apparatus in claim 1 further comprising a clip configured to substantially hold the detachable nozzle manifold in place on the mask.

3. The apparatus in claim 1 further comprising an adhesive layer configured to substantially hold the detachable nozzle manifold in place on the mask.

4. The apparatus of claim 1 further comprising a flexible conduit communicating the pressurised gases to the inlet.

5. The apparatus of claim 4 further comprising a quick release connector between the flexible conduit and the inlet.

6. The apparatus of claim 1 further comprising a filtered blower configured to deliver filtered pressurised air to the inlet.

7. The apparatus of claim 6 wherein the blower is variable speed configured to deliver 20-30 L/min.

8. The apparatus of claim 1 wherein the seal comprises a first elongated foam strip adhered to the side of the manifold configured to face the user, and a second elongated foam strip shorter than the first strip adhered to the side of the manifold configured to attach to the mask.

9. The apparatus of claim 1 wherein the mask is N95 certified disposable face mask.
